(19) United States
(12) Patent Application Publication
(10) Pub. No.: US 2020/0216870 A1
(43) Pub. Date: Jul. 9, 2020

(54) NON-PURIFIED NUCLEIC ACID AMPLIFICATION METHOD AND DEVICE

(71) Applicant: ZHUHAI ASTROBIO BIOTECHNOLOGY CO, LTD., Zhuhai (CN)

(72) Inventors: Xing SU, Zhuhai (CN); Kaiyuan WU, Zhuhai (CN)

(21) Appl. No.: 16/648,320

(22) PCT Filed: Sep. 14, 2018
(86) PCT No.: PCT/CN2018/105635

§ 371 Date: Mar. 18, 2020

(30) Foreign Application Priority Data
Sep. 18, 2017 (CN) 201710841430.X

(51) Int. Cl. C12P 19/34; B01L 3/00

(52) U.S. Cl. CPC C12P 19/34; B01L 3/508; B01L 3/565; B01L 2300/161; B01L 2300/041; B01L 2300/0832; B01L 2300/06; B01L 2200/16

(57) ABSTRACT

The invention relates to a method and device for amplifying non-purified nucleic acid, where the method comprises the following steps: Providing a reaction device in which an amplification reaction reagent is placed; Sampling with a sampler: the sampler includes a sealing block and a sample needle connected with the sealing block. The end of the sample needle is provided with a hydrophilic surface, which contacts the non-purified nucleic acid sample for sampling; the hydrophilic surface is inserted into the amplification reaction reagent, and the reaction device is sealed by the sealing block; The temperature of the reaction device is controlled by the temperature control instrument to carry out the amplification reaction. The device of the invention is used for realizing the method. The invention can save the process of sample preparation and purification, directly amplify the non-purified sample, reduce the operation difficulty, save the time and reduce the cost.

### TECHNICAL FIELD

The invention relates to the field of molecular biology, in particular to a method for the qualitative or quantitative determination of nucleic acid.

### BACKGROUND OF THE INVENTION

With the development of biotechnology, modern molecular biology technology or genetic engineering technology is increasingly widely used in various biotechnology industries, especially in medical diagnosis. The application of these techniques often involves the qualitative and quantitative determination with molecular probes. For example, when determining the nucleic acid (DNA or RNA) samples of animals and plants, the nucleic acid in the samples needs to be purified first, and then the nucleic acid amplification reaction is carried out. The typical nucleic acid amplification reaction is polymerase chain reaction (PCR) or reverse transcriptase polymerase chain reaction (RT-PCR).

However, the purification of nucleic acid is a complex process. For different sample sources, different purification methods are needed. The common purification methods include magnetic particle method, organic extraction and precipitation method. The purification process of nucleic acid requires the use of additional consumables and devices, which not only takes time, but also increases the difficulty and cost of use. In addition, when the existing purification methods are used to purify the small amount of nucleic acid samples, the recovery rate of nucleic acid is low, which easily leads to unreliable detection results.

### SUMMARY OF THE INVENTION

In order to solve the above problems, the main purpose of the invention is to provide a method for directly amplifying non-purified nucleic acid samples. Another purpose of the invention is to provide a device for directly amplifying an unpurified nucleic acid sample.

In the invention, the unpurified nucleic acid sample is the unpurified or incompletely purified nucleic acid sample in natural or non-natural existence, and also includes the biological sample containing nucleic acid, such as animal tissue (muscle, blood, etc.), plant tissue (leaf, stem, root, etc.), animal and plant products, original animal and plant samples, products, secretion or excretion, etc. Non-purified nucleic acid samples also include natural or artificial samples (water, soil, air, etc.) containing organisms (eukaryotes, prokaryotes, viruses, etc.).

Amplification is a chemical or biological enzyme reaction that can increase the number of molecules, or complementary molecules, or related molecules. Biological enzyme reaction includes one or more combinations of polymerase chain reaction (PCR), reverse transcriptase polymerase chain reaction (RT-PCR), multiplex polymerase chain reaction, quantitative polymerase chain reaction (QPCR) and nested PCR.

In order to achieve the above main purpose, the invention provides a method for amplifying non-purified nucleic acid, which comprises the following steps: providing a reaction device in which an amplifying reaction reagent is placed; using a sampler for sampling: The sampler includes a sealing block and a sampler needle connected with the sealing block. The end of the sampler needle is provided with a hydrophilic surface, which contacts the non-purified nucleic acid sample through the hydrophilic surface for sampling; the hydrophilic surface is inserted into the amplification reaction reagent, and the reaction device is sealed by the sealing block; the temperature of the reaction device is controlled by the temperature control device, and the amplification reaction is carried out.

The invention uses the reaction device in combination witha sampler with a sealing block, to carry out sampling, sample adding and nucleic acid amplification reaction under the closed condition. The end of the sampler needle for sampling and sample adding has a hydrophilic surface, which can directly contact with a liquid or a solid, so that the hydrophilic surface would carry the sample. The hydrophilic surface can be a radial surface at the lower end of the sampler needle. When the hydrophilic surface of the sampler needle is inserted into the amplification reaction reagent, the attached sample will be separated from the hydrophilic surface and released to the amplification reaction reagent as the source of the nuclear acid substrate for the nucleic acid amplification reaction. Because the hydrophilic surface of the sampler needle contains small amount of samples thus less impurities along with the sample, it will not interfere with the amplification reaction. The method of the invention eliminate the process of sample preparation and purification, directly amplifies the non-purified sample, reduces the operation complexity, saves the operation time and reduces the cost.

The reaction device used by the method of the invention can include a tubular chamber in which an amplification reaction reagent is placed. The amplification reaction reagent can be added manually or automatically before the use of the reaction device, or added in advance, and stored or transported after being sealed. When using, the reaction device only needs to be opened to add samples, especially suitable for use by individuals or small work settings.

The sampler needle is connected with the sealing block. The sealing block can be in the form of a cover or a plug to work with the reaction device. Specifically, it can work withthe opening or through hole of the reaction device to achieve the sealing of the reaction device and avoid the pollution caused by the reaction reagent or product.

The temperature of the reaction device can be controlled by all known means. The temperature of the reaction device can be in overall equalibrium, or overall temperature change in an equalbrium or maintaining certain temperature difference across the different parts e such as maintaining a temperature gradient. The temperature control mode can be constant heating of specific parts of the reaction device, or maintaining a constant temperature gradient, or temperature control with cyclic changes, so that the reaction device has a homogeneous change of temperature in a cyclic fashion. The effect is to make the molecules in the tubular chamber undergo different temperatures, so as to meet the requirements of different enzyme reaction conditions and achieve the purpose of nucleic acid amplification in the tubular chamber.

In one embodiment that the hydrophilic surface is not completely smooth.

The hydrophilic surface of the sampler needle can be achieved by existing known methods, for example, some or all surfaces of the sampler needle can be modified or modified by organic or inorganic chemical methods to obtain hydrophilicity. As one of the simple methods, the surface of the lower end of the sampler needle can be machined into a non-perfectly smooth surface, thus increasing the effective surface area.

In another emobodiment, the sampler needle can move up and down through the sealing block; before the amplification reaction, the sampler needle moves up relative to the sealing block, and the hydrophilic surface leaves the amplification reaction reagent.

The sampler needle is connected with the sealing block in a fixed or movable way. As a movable way, the sampler needle preferably penetrates the sealing block and can move up and down in the sealing block. When the sampler needle is added to the amplification reaction reagent, most of the sampler needle is located under the sealing block to ensure enough length to send the sample on the hydrophilic surface into the amplification reaction reagent. When the sample addition is completed, the sampler needle can be moved upward without moving the sealing block to ensure that the reaction system is sealed and the sampler needle can be moved from the amplification reaction reagent at the same time, so as to prevent possible interference to the reaction and subsequent signal detection.

In another embodiment, the reaction device contains multiple tubular chambers, wherein amplification reaction reagents are placed in all tubular chambers ; using a single or multiple samplers for sampling of one or more unpurified nucleic acid samples to carry out multiple amplification reactions.

The reaction device of the invention can have one or more chambers for amplification reaction. When multiple amplification reactions are needed, the reaction device with multiple tubular chambers can be used, and the amplification reaction reagent can be added in multiple tubular chambers. Tubular chambers can be connected or separated from each other through channels. With the combination of multiple samplers and multiple tubular chambers, multiple amplification reactions can be carried out at the same time or at different times, further improving the efficiency. Another emobiment is that the amplification reaction reagent includes an polymerase with anti-interference properties.

Amplification reaction reagents include polymerase, nucleotide, buffer and other conventional reagents for amplification reaction. Among them, a polymerases with anti-interference properties are preferred, which can resist the interference of impurities and further ensure the stable and reliable amplification. The polymerases with anti-interference properties can be obtained from available commercial vendors, such as Taq DNA polymerases derivatives (Hemo KlenTaq, BIOTAQ, etc.) or other DNA polymerase derivatives Phusion and Phir polymerases.

In another embodiment, the amplification reaction reagent also includes molecular probe or affinity substance; the method also includes detecting the signal of amplification reaction for qualitative or quantitative analysis.

Molecular probes or affinity substances can be added to the amplification reagents. Through the combination of molecular probes or affinity substances with the reaction products of biological enzymes, optical or electrical signals related to the product quantity can be obtained. Optical signal includes fluorescence signal, light absorption signal, infrared absorption signal, Raman scattering signal, chemiluminescence signal, etc. Among them, affinity substances refer to substances that can be directly combined with products, such as dyes and nanoparticles. In the process of amplification reaction, the signal can be detected, and the results can be analyzed qualitatively and quantitatively by the existing known methods. To achieve the another stated purpose above, the invention provides a non-purified nucleic acid amplification device, which includes a reaction device, wherein an amplification reaction reagent is placed; a sampler, wherein the sampler includes a sealing block which can be closed and worked with the reaction device, and a sampler needle which is connected with the sealing block; the end of the sampler needle is provided with a hydrophilic surface.

The device provided by the invention includes a reaction device and a sampler. The sealing block in the sampler works with the reaction device to realize the sealing of the reaction device and avoid the pollution caused by the reaction reagent or product. The sampler also includes a sampler needle connected with a sealing block, the end of the sampler needle has a hydrophilic surface, which can directly contact with a liquid or a solid, so that a small amount of samples are attached to the hydrophilic surface. When adding samples, the sampler is inserted into the reaction device, and the sealing block is sealed with the reaction device. At the same time, the samples on the hydrophilic surface enter the amplification reaction reagent as the source of the nucleic acid substrate for the nucleic acid amplification reaction. Because the hydrophilic surface of the sampler needle has less samples and less impurities, it will not interfere with the amplification reaction. The device can realize the above method, save the process of sample preparation and purification, and directly amplify the non-purified samples.

In some embodiments, the sampler needle is made of metal or other or non-metallic materials, with a diameter between 0.1 and 5 mm, and the ratio of length to diameter is greater than or equal to 3; the hydrophilic surface is a non-perfectly smooth surface.

The sampler needle can be made of metal or other materials. When the diameter and length of the sampler needle are within the above range, the sample addition requirements can be met.

The further technical solution is that the sampler needle is fixedly connected with the sealing block, or the sampler needle can move up and down through the sealing block.

The sampler needle can be fixedly connected with the sealing block. After sample addition, the sampler needle can be left in the reaction tube. The sampler needle can also be connected with the sealing block in a movable way. For example, the sampler needle can move up and down through the sealing block. After sample addition, the sampler needle can be moved to pull the sampler needle out of the reaction area, or take the sampler needle out of the reaction tube, but still keep the sealing of the reaction tube.

In some embodiements, the reaction device comprises one or more tubular chambers containing amplification reaction reagents.

The reaction device may have one or more tubular chambers which may be connected or separated from each other through channels. Multiple amplification reactions can be carried out simultaneously or not at the same time by using multiple tubular chambers.

### DESCRIPTION OF FIGURES

FIG. 1 is a schematic diagram of a sampler structure according to the first example of the present invention.

FIG. 2 is a structural diagram of the first example of the present invention.

FIG. 3 is a schematic diagram of the sampler structure of the second example of the present invention.

FIG. 4 is a structural diagram of the second example of the present invention.

### SPECIFIC IMPLEMENTATION

This invention of the method for amplification ofnon-purified nucleic acid is a general method, which is suitable for the solid or liquid samples as source of a variety of animal and plant nucleic acids, especially useful when there is small amount of sample . In practical application, according to different kinds of nucleic acids, suitable polymerases, nucleotides, buffers, primers, molecular probes, affinity substances, etc. can be selected as amplification reaction reagents, placed in reaction containers, and then combined with the sampler of the invention, amplification reaction can be carried out under closed conditions.

The method and device of the invention will be further described below with the figures and examples.

### THE FIRST EXAMPLE

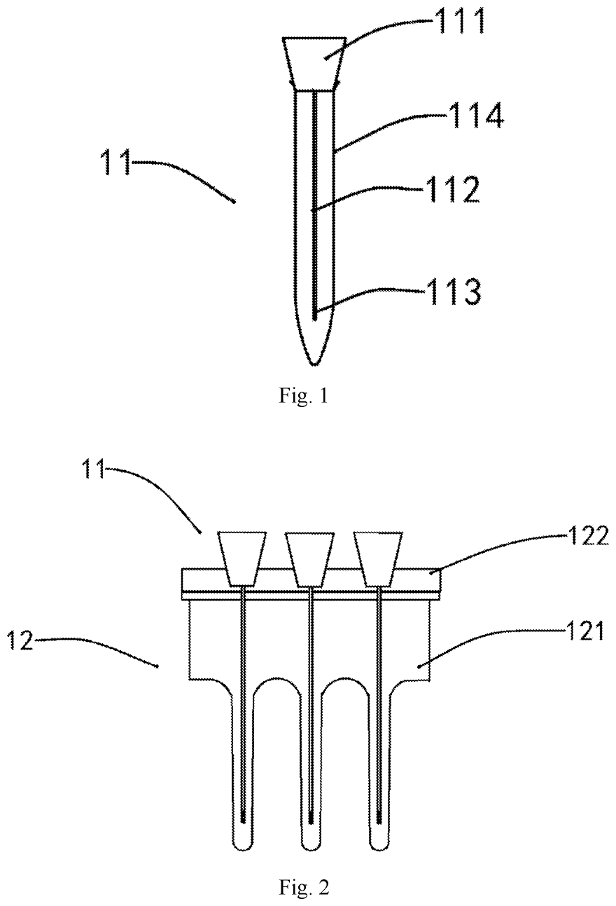

The device used in this example is shown in FIGS. 1 to 2, which includes a sampler 11 and a reaction device 12.

The sampler 11 includes a sealing block 111 and a sampler needle 112 fixedly connected with the sealing block 111. The sealing block 111 is in the form of a plug. The sampler needle 112 is made of metal with a diameter between 0.1 and 5 mm and a length to diameter ratio greater than or equal to 3. The radial surface of the lower end of the sampler needle 112 is rough, forming a hydrophilic surface 113. In this example, the sampler 11 also includes a protective sleeve 114. Before the sampler needle 112 is used, the sampler needle 112 is inserted into the protective sleeve 114 for protection to prevent the sampler needle 112 from being polluted.

The reaction device 12 may include a reaction vessel 121 and a cover body 122. The reaction vessel 121 and the cover body 122 form a sealed device. The cover body 122 can be provided with a through hole, and the sealing block 111 can be inserted into the through hole for sealing. The reaction vessel 121 may have tubular chambers for carrying out amplification reactions. The tubular chamber may be connected or unconnected. There are amplification reagents in the tubular chamber.

When the device is used for the amplification reaction of the unpurified nucleic acid, the protective sleeve 114 on the sampler 11 is removed first, the end of the sampler needle 112 is contacted with the unpurified nucleic acid sample, and a small amount of sample is dipped. As shown in FIG. 2, the sampler 11 is inserted into the reaction device 12, at this time, the hydrophilic surface 113 contacts with the amplification reaction reagent, and the sample inserts into the amplification reaction reagent as the source of the amplification reaction substrate. At the same time, the sealing block 111 is worked with the cover body 122 to form a sealing structure.

Then the amplification reaction is carried out. Generally, the enzyme reaction is between 15° C. and 99° C. Temperature control of biological enzyme reaction can be carried out by using currently known methods, such as infrared light, hot/cold wind, cold/hot solid or liquid substances, electromagnetic induction, etc. The closed reaction device 12 can be inserted into the temperature control device for reaction. According to the requirements of the reaction, any tubular chamber can withstand constant temperature or periodic change temperature, and the tubular chamber can also have equilibrium temperature or gradient temperature. For example, similar to the traditional PCR temperature control method, the temperature of the temperature control device changes periodically under the control of a computer program, such as holding for several seconds to several minutes under a certain temperature, and the tubular chamber is completely inserted into the heating part of the temperature control device, during which the liquid temperature in the tubular chamber is basically homogeneous. In another example of the gradient temperature control methodwith constant temperature, the temperature of the temperature control device remains unchanged under the control of the computer program, and only part of the tubular chamber contacts the heating part of the temperature control device. When the bottom is heated, the bottom temperature will be higher than the top temperature, and the liquid in the tubular chamber will have a temperature gradient. Because the liquid with low temperature in the upper part has relatively high density or specific gravity, the liquid in the upper part and the liquid in the lower part will produce convection. The effect is to drive the molecular flow in the tubular chamber, and to undergo different temperatures, so as to meet the requirements of different enzyme reaction conditions and achieve the purpose of nucleic acid amplification in the tubular chamber.

By detecting the signal response of the reaction, qualitative and quantitative analysis of the results can be carried out with known methods.

### EXAMPLE 2

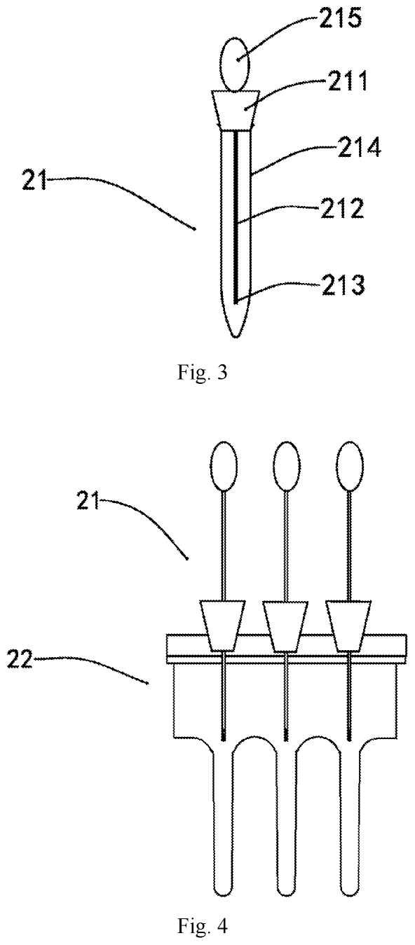

The device used in this example is shown in FIGS. 3 to 4, which includes a sampler 21 and a reaction device 22. The sampler 21 includes a sealing block 211, a sampler needle 212 and a protective sleeve 214, and a hydrophilic surface 213 is placed at the lower end of the sampler needle 212. The sampler needle 212 penetrates the sealing block 211 and moves up and down along the sealing block 211. The upper end of the sampler needle 212 is also provided with a pull block 215 for lifting the sampler needle 212 upward. The remaining structures of the device are the same as those in the first example. When the device of the example is used for the amplification reaction of the unpurified nucleic acid, the steps are basically the same as those of the first example. The difference is that, as shown in FIG. 4, after the sample addition is completed, the pull block 215 is moved to so that sampler needle 212 can move upward, so as to remove the sampler needle 212 from the reaction system.

It can be seen from the above that the method and device of the invention can save the process of sample preparation and purification, directly amplify the non-purified sample, reduce the operation difficulty, save the operation time and reduce the cost.

Finally, it should be emphasized that the above is only a preferred example of the invention and is not used to limit the invention. For those skilled in the art, the practice of this invention may have different variations. Any modification, equivalent replacement and improvement made within the spirit and principles of the invention shall be included in the protection scope of the invention.

1-10. (canceled)

11. A non-purified nucleic acid amplification method comprises:
placing an amplification reaction reagent in a reaction device;
sampling with an apparatus by contacting a hydrophilic surface of a sample needle with a non-purified nucleic acid sample, wherein the apparatus comprises a sealing block and the sample needle connected with the sealing block, an end of the sample needle is provided with the hydrophilic surface;
inserting the hydrophilic surface into the amplification reaction reagent;
sealing the reaction device through the sealing block;
performing an amplification reaction in the reaction device wherein a temperature of the reaction device is controlled by a temperature control instrument.

12. The non-purified nucleic acid amplification method of claim 11, wherein the hydrophilic surface is not completely smooth.

13. The non-purified nucleic acid amplification method of claim 11, wherein the sample needle move up and down through the sealing block, the sample needle is upward relative to the sealing block and the hydrophilic surface from the amplification reaction reagent is removed before the amplification reaction.

14. The non-purified nucleic acid amplification method of claim 11, wherein the reaction device comprises a tubular chamber comprising the amplification reaction reagent.

15. The non-purified nucleic acid amplification method of claim 11, wherein the amplification reaction is a PCR, a RT-PCR, a nested PCR, an quantitative PCR, a multiplex PCR or combinations thereof.

16. The non-purified nucleic acid amplification method of claim 11, wherein the non-purified nucleic acid sample is a natural or a non-natural sample containing a nucleic acid; a signal is detected of the amplification reaction for qualitative or quantitative analysis.

17. The non-purified nucleic acid amplification method of claim 11, wherein the amplification reaction reagent comprises a polymerase, a nucleotides, a buffer for the amplification reaction.

18. The non-purified nucleic acid amplification method of claim 17, wherein the polymerase has an anti-interference property.

19. A non-purified nucleic acid amplification device comprises:
a reaction device comprising an amplification reaction reagent;
an apparatus comprising (a) a sealing block to close and match with the reaction device, and (b) a sample needle in configuration with connected with the sealing block; an end of the sample needle comprises a hydrophilic surface.

20. The non-purified nucleic acid amplification device of claim 19, wherein the sample needle is made of a metal or a non-metal, with a diameter of 0.1 mm to 5 mm and a ratio of length to diameter is greater than or equal to 3; wherein the hydrophilic surface is not completely smooth.

21. The non-purified nucleic acid amplification device of claim 19, wherein the sample needle either fixedly connected with the sealing block, or the sample needle move up and down through the sealing block.

22. The non-purified nucleic acid amplification device of claim 19, wherein the reaction device comprises a tubular chamber comprising the amplification reaction reagent.

23. An apparatus comprises:
a sealing block;
a needle configured with the sealing block;
a protective sleeve configured to protect the needle;
an end of the needle comprises a hydrophilic surface;
wherein the hydrophilic surface carries a small amount of a sample.

24. The apparatus of claim 23, wherein the hydrophilic surface is not smooth.

25. The apparatus of claim 23, wherein, the needle is configured to move up and down through the sealing block.

26. The apparatus of claim 23, wherein the protective sleeve on the apparatus is removable to contact the sample with the end of the needle, and the apparatus is inserted into a reaction device comprising an amplification reaction reagent.

27. The apparatus of claim 26, wherein the hydrophilic surface is configured to insert into the amplification reaction reagent to separate the sample.

28. The apparatus of claim 23, wherein the sealing block comprises a plug form.

29. The apparatus of claim 23, wherein the needle is made of a metal or a non-metal, with a diameter of 0.1 mm to 5 mm and a ratio of length to diameter is greater than or equal to 3.

30. The apparatus of claim 23, wherein the apparatus is used in an amplification of a non-purified nucleic acid.
